(19) United States
(12) Patent Application Publication
(10) Pub. No.: US 2009/0312650 A1
(43) Pub. Date: Dec. 17, 2009

(54) IMPLANTABLE PRESSURE SENSOR WITH AUTOMATIC MEASUREMENT AND STORAGE CAPABILITIES

(75) Inventors: Keith R. Maile, New Brighton, MN (US); Abhijeet V. Chavan, Maple Grove, MN (US); Paul J. Huelskamp, St. Paul, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(73) Assignee: CARDIAC PACEMAKERS, INC., St. Paul, MN (US)

(21) Appl. No.: 12/464,508

(22) Filed: May 12, 2009

Related U.S. Application Data
(60) Provisional application No. 61/060,877, filed on Jun. 12, 2008.

(51) Int. Cl. A61B 5/02

(52) U.S. Cl. 600/486

(57) ABSTRACT

Methods for activating implantable medical devices within a patient's body are disclosed. An illustrative method includes activating an implantable medical device from a low-power state to an awake state in response to a scheduled time event, sensing one or more pressure measurements within the body, computing an average pressure measurement based on the sensed pressure measurements, storing the average pressure measurement within a memory of the implantable medical device, and then returning the device to the low-power state. A triggering event such as the detection of patient activity or motion can also be used to activate the implantable medical device between the low-power state and an active state.

### CROSS-REFERENCE TO RELATED APPLICATION

This application claims priority under 35 U.S.C. § 119 to U.S. Provisional Application No. 61/060,877, filed on Jun. 12, 2008, entitled “Implantable Pressure Sensor With Automatic Measurement and Storage Capabilities,” which is incorporated herein by reference in its entirety for all purposes.

### TECHNICAL FIELD

The present invention relates generally to implantable medical devices. More specifically, the present invention pertains to methods for activating implantable medical devices within the body.

### BACKGROUND

Implantable medical devices (IMDs) such as pacemakers and implantable cardioverter defibrillators are utilized in monitoring and regulating various conditions within the body. An implantable cardioverter defibrillator, for example, may be utilized in cardiac rhythm management applications to monitor the rate and rhythm of the heart and for delivering various therapies such as cardiac pacing, cardiac defibrillation, and/or cardiac therapy. In some cases, the implantable medical device can be configured to sense various physiological parameters occurring within the body to determine the occurrence of any abnormalities in the operation of the patient's heart. Based on these sensed parameters, the implantable medical device may then deliver an appropriate treatment to the patient.

Communication with implantable medical devices is often accomplished via a telemetry link between an external device and the implanted medical device, or between the implanted medical device and another device located within the body. Establishing and maintaining a communications link between the implanted medical device and the external device or other communicating device is often energy consuming, which can drain the power supply and shorten the operational life of the device.

### SUMMARY

The present invention pertains to methods for activating implantable medical devices within the body. An illustrative method carried out by an implantable medical device includes activating the device from a low-power or sleep state to an active state at a scheduled time event programmed within the device, sensing one or more pressure measurements within the body, computing an average pressure measurement based on the one or more sensed pressure measurements, storing the average pressure measurement within a memory of the device, and then returning the device to the low-power state.

In some embodiments, the implantable medical device is configured to store several average pressure measurements within memory and then later transmit the measurements to another device in communication with the device for reconstruction and analysis. Alternatively, and in other embodiments, the implantable medical device is configured to simultaneously transmit the average pressure measurements to another device, allowing the measurements to be analyzed in real-time. In certain embodiments, a triggering event can be configured to prompt the implantable medical device to activate and take one or more measurements within the patient's body.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of an illustrative system employing a remote sensor located within the body;

FIG. 2 is a block diagram of the remote sensor of FIG. 1;

FIG. 3 is a flow chart showing an illustrative method of activating an implantable medical device;

FIG. 4 is a graph showing the operating current of an implantable medical device over multiple activation cycles;

FIG. 5 is a graph showing an illustrative method of taking average pressure measurements from a pressure waveform sensed by an implantable medical device over a cardiac cycle; and

FIG. 6 is a flow chart showing another illustrative method of activating an implantable medical device.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

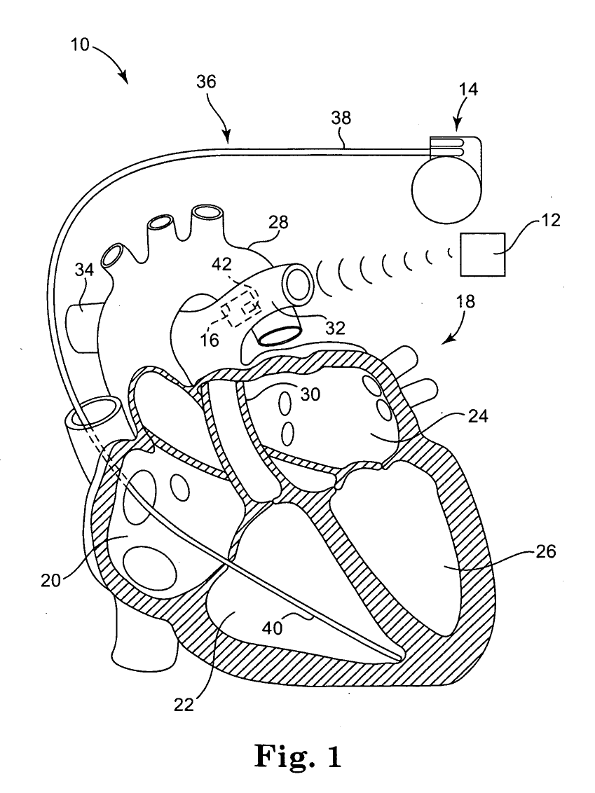

FIG. 1 is a schematic view of an illustrative system 10 employing a remote sensor located within the body of a patient. The system 10, illustratively a cardiac rhythm management system for providing cardiac rhythm management to a patient, includes an external monitor 12 (e.g., an external wand or programmer), a pulse generator 14 implanted within the body at a location below the patient's skin, and a remote sensor 16 implanted deeply within the patient's body such as in one of the arteries or ventricles of the patient's heart 18, or in one of the vessels leading into or from the heart 18. The heart 18 includes a right atrium 20, a right ventricle 22, a left atrium 24, a left ventricle 26, and an aorta 28. The right ventricle 22 leads to the main pulmonary artery 30 and the branches 32,34 of the main pulmonary artery 30. Typically, the pulse generator 14 will be implanted at a location adjacent to the location of the external monitor 12, which may lie adjacent to the exterior surface of the patient's skin.

In the illustrative CRM system 10 depicted, the pulse generator 14 is coupled to a lead 36 deployed in the patient's heart 18. The pulse generator 14 can be implanted subcutaneously within the body, typically at a location such as in the patient's chest or abdomen, although other implantation locations are possible. A proximal portion 38 of the lead 36 can be coupled to or formed integrally with the pulse generator 14. A distal portion 40 of the lead 36, in turn, can be implanted at a desired location within the heart 18 such as the right ventricle 22, as shown. Although the illustrative system 10 depicts only a single lead 36 inserted into the patient's heart 18, it should be understood, however, that the system 10 may include multiple leads so as to electrically stimulate other areas of the heart 18. In some embodiments, for example, the distal portion of a second lead (not shown) may be implanted in the right atrium 20. In addition, or in lieu, another lead may be implanted at the left side of the heart 18 (e.g., in the coronary veins) to stimulate the left side of the heart 18. Other types of leads such as epicardial leads may also be utilized in addition to, or in lieu of, the lead 36 depicted in FIG. 1.

During operation, the lead 36 is configured to convey electrical signals between the heart 18 and the pulse generator 14. For example, in those embodiments where the pulse generator 14 is a pacemaker, the lead 36 can be utilized to deliver electrical therapeutic stimulus for pacing the heart 18. In those embodiments where the pulse generator 14 is an implantable cardiac defibrillator, the lead 36 can be utilized to deliver electric shocks to the heart 18 in response to an event such as a heart attack. In some embodiments, the pulse generator 14 includes both pacing and defibrillation capabilities.

The remote sensor 16 can be configured to perform one or more designated functions, including the sensing of one or more physiological parameters within the body. Example physiological parameters that can be measured using the remote device 16 can include, but are not limited to, blood pressure, blood flow, temperature, and strain. Various electrical, chemical, magnetic and/or sound properties may also be sensed within the body via the remote sensor 16.

In the exemplary embodiment of FIG. 1, the remote sensor 16 comprises a pressure sensor implanted at a location deep within the body such as in the main pulmonary artery 30 or a branch 32,34 of the main pulmonary artery 30 (e.g., in the right or left pulmonary artery). An exemplary pressure sensor suitable for use in sensing pulmonary arterial pressure is described in U.S. Pat. No. 6,764,446, entitled “Implantable Pressure Sensors and Methods for Making and Using Them,” which is incorporated herein by reference in its entirety for all purposes. In use, the pressure sensor 16 can be used to predict decompensation of a heart failure patient and/or to aid in optimizing pacing and/or defibrillation therapy via the pulse generator 14 by taking pressure measurements within the body. In some embodiments, the pressure sensor 16 can be configured to sense, detect, measure, calculate, or derive other associated parameters such as flow rate, maximum and minimum pressure, peak-to-peak pressure, rms pressure, and/or pressure rate change, as discussed further herein. In some embodiments, the absolute pressure measurements taken by the remote sensor 16 can be referenced against barometric pressure in order to derive gauge pressure values.

The remote sensor 16 may be implanted in other regions of the patient's vasculature, in other body lumens, or in other areas of the body, and may comprise any type of chronically implanted device or remote device adapted to deliver therapy or monitor biological and chemical parameters, properties, and functions. The remote sensor 16 can be tasked, either alone or with other implanted or external devices, to provide various therapies within the body. In certain embodiments, for example, the remote sensor 16 may comprise a glucose level sensor that can be used in conjunction with an insulin pump for providing insulin treatment to the patient. Although a single remote sensor 16 is depicted in FIG. 1, multiple such devices could be implanted at various locations within the body for sensing physiologic parameters at multiple regions within the body. In some embodiments, for example, multiple remote sensors may be implanted throughout the body, and can be configured to wirelessly communicate with each other, the external monitor 12, the pulse generator 14, and/or with other devices located inside or outside of the body.

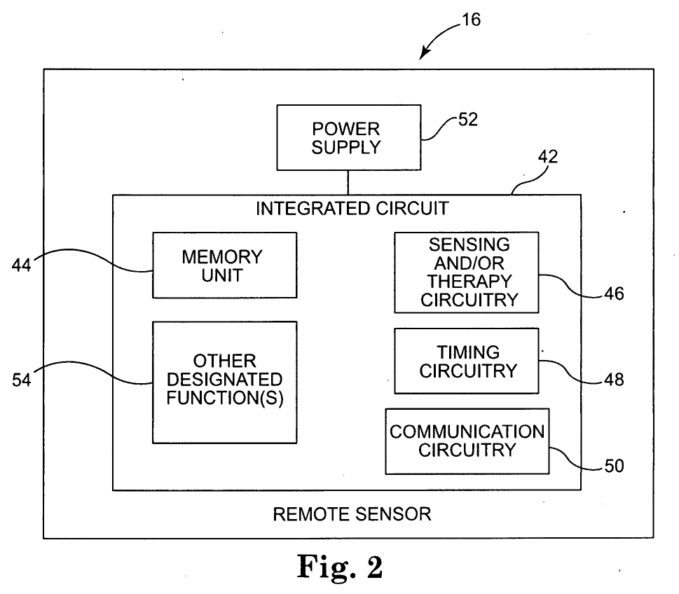

FIG. 2 is a block diagram showing several illustrative components of the remote sensor 16 of FIG. 1. In the embodiment of FIG. 2, the remote sensor 16 includes an integrated circuit (IC) 42, which contains a memory 44, sensor and/or therapy circuitry 46, timing circuitry 48, and communication circuitry 50. A power supply 52 such as a battery or power capacitor is electrically connected to the integrated circuit 42 for use in powering the remote sensor 16.

The integrated circuit 42 can comprise a digital signal processor, a microprocessor, an application-specific integrated circuit (ASIC), or other suitable hardware adapted to facilitate sensing, therapy delivery, as well as the performance of one or more other designated functions 54. The integrated circuit 42 may execute software resident in memory 44. In some embodiments, the memory 44 may comprise a volatile or non-volatile memory unit, and includes a data table containing timing data that the integrated circuit 42 uses to program the timing circuitry 48. For example, the data table may contain values representing designated time interval(s) in which one or more components of the remote sensor 16 wake-up or become activated.

The steps and functionality to be performed by the remote sensor 16 may be embodied in machine-executable instructions operating on a software and/or hardware platform. In some embodiments, for example, the instructions may be embodied in a processor or controller to perform the steps. In other embodiments, the various steps may be performed by specific hardware components that contain logic for performing the steps, or by a combination of programmed computer components and custom hardware components. In some embodiments, the remote sensor 16 includes firmware containing updatable instructions to be used by the sensor 16.

The sensing and/or therapy circuitry 46 performs functions related to the measurement of physiology parameters and/or therapy. Examples of possible physiologic measurements include, but are not limited to, blood pressure, temperature, blood or fluid flow, respiratory rate, strain and various electrical, chemical, magnetic, and/or sound properties within the body. In some embodiments, for example, the sensing and/or therapy circuitry 46 could be used to sense lung health, heart valve operation, irregular flow, etc. by sensing the presence of sounds within the body. Examples of therapeutic functions include, but are not limited to, providing heart pacing therapy, cardiac defibrillation therapy, cardiac resynchronization therapy, and/or drug delivery therapy. In some embodiments, the sensing and/or therapy circuit 46 can include an activity sensor for measuring patient activity, an accelerometer for monitoring body posture or orientation, a temperature sensor for measuring body temperature, and/or a respiratory sensor for monitoring respiratory rhythms.

The timing circuitry 48 performs functions related to the scheduling, prompting, and activating of various activities to be performed by the remote sensor 16. In some embodiments, the timing circuitry 48 employs low-power, internal timers or oscillators to coordinate the activation of selective components of the remote sensor 16 using a timing reference. Examples of timing references that utilize a low amount of power are oscillators, such as RC relaxation, LC tuned circuit, and crystal stabilized oscillators.

The communication circuitry 50 includes circuitry that allows the remote sensor 16 to communicate with the external monitor 12, the pulse generator 14, other implanted sensors, and/or other devices located inside or outside of the body. In some embodiments, for example, the communication circuitry 50 includes circuitry that allows the remote sensor 16 to wirelessly communicate with other devices via a wireless telemetry link. Example modes of wireless communication can include, but are not limited to, acoustic, radio frequency, inductive, optical, or the like.

At certain time periods, selected components of the remote sensor 16 may be powered off in a low-power or sleep state in order to conserve energy usage from the power supply 52. As discussed further herein, the timing circuitry 48 can cause selected components of the remote sensor 16 to wake-up or become active at scheduled times programmed within memory 44. For example, the sensing and/or therapy circuitry 46 may be activated at one or more scheduled times to perform designated therapy and/or sensing functions within the body. The timing circuitry 48 can also prompt the activation of other sensor components such as the communication circuitry 50 to permit measurements to be transmitted to other communicating devices in either real-time, or to permit the transmission of stored measurements at a later time.

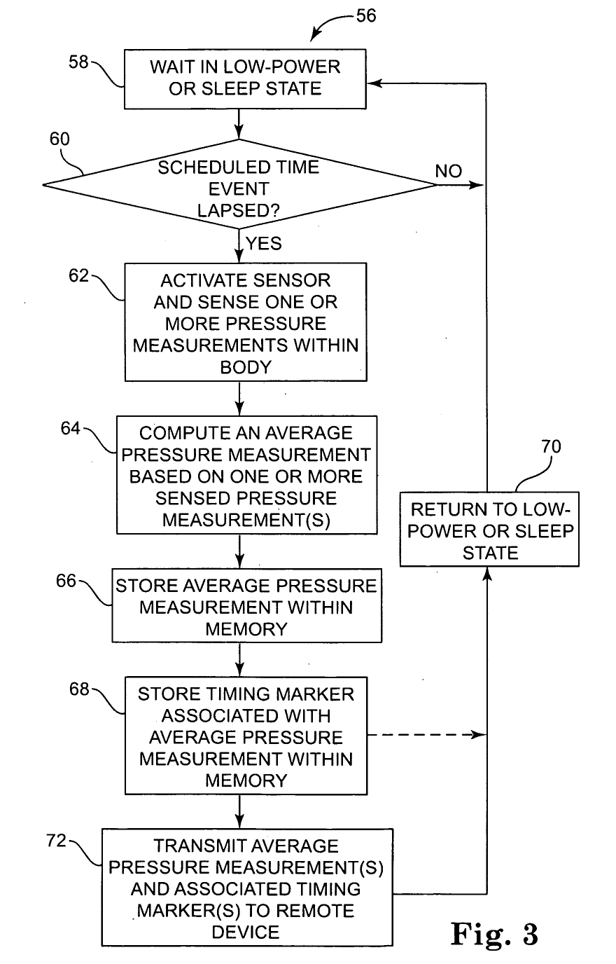

FIG. 3 is a flow chart showing an illustrative method 56 of activating an implantable medical device such as the remote sensor 16 of FIG. 1. To conserve energy usage from the power supply 52, the remote sensor 16 initially waits (block 58) in a low-power or sleep state in which selective components of the remote sensor 16 are either deactivated or placed in a low-power mode. In certain embodiments, for example, only the timing circuitry 48 within the remote sensor 16 is activated during the low-power or sleep state, thus reducing the power demand associated with continuously operating the sensing and/or therapy circuitry 46 and the communications circuitry 50. Other components of the remote sensor 16 may also be activated during the low-power or sleep state. In those embodiments in which the memory 44 is a non-volatile memory, for example, the remote sensor 16 may provide power necessary to maintain the contents stored within the memory 44.

From the low-power or sleep state (block 58), the timing circuitry 48 is configured to determine whether a scheduled timing event stored in memory 44 has lapsed (block 60). The determination of whether a scheduled timing event has lapsed can be accomplished, for example, by the timing circuitry 48 calling a table of scheduled operating times pre-programmed within memory 44 and comparing the scheduled operating times against the current time and/or date to determine if a scheduled time event has occurred. If the timing circuitry 48 determines that a scheduled time event has not lapsed, the remote sensor 16 continues operation in the low-power or sleep state (block 58). Otherwise, if the timing circuitry 48 determines that a scheduled time event has lapsed, the timing circuitry 48 then activates (block 62) the sensing and/or therapy circuitry 46 within the remote sensor 16, causing the sensor 16 to activate and take one or more measurements within the body. In those embodiments in which the remote sensor 16 is a pressure sensor implanted in an artery, for example, the pressure sensor may activate a pressure sensing transducer and sense one or more pressure measurements within the artery. To conserve power, the remote sensor 16 may activate only those components necessary to take and store the pressure measurements.

Once the sensing and/or therapy circuitry 46 is activated and a number of measurements have been taken within the body, the remote sensor 16 can then be configured to compute or extract an average pressure measurement (block 64) based on the one or more sensed measurements, and then store the average pressure measurement within memory 44 (block 66). In some embodiments, computation or extraction of an average pressure measurement can be accomplished by sampling pressure measurements from a pressure waveform measured by the remote sensor 16 over a cardiac cycle, and then storing an average pressure measurement within memory 44 representative of the actual pressure during the cycle. The computation or extraction of average pressure measurements can be accomplished, for example, by sampling pressure measurements at discrete time intervals, by computing an ongoing mean pressure measurement that is updated by each subsequent pressure measurement taken, by selectively taking only certain measurements (e.g., only those pressure measurements that are above or below a particular threshold), by taking peak-to-peak measurements, and so forth.

In use, the computation and storage of average pressure measurements in lieu of actual pressure measurements reduces the amount of data storage required by the remote sensor 16, thus reducing the size of memory 44 required. In some cases, the computation and storage of average pressure measurements may permit the remote sensor 16 to take measurements over a longer period of time before an outward transmission of the measurements is necessary. For instance, in some embodiments the ability to store average pressure measurements within memory instead of the entire pressure waveform allows the remote sensor 16 to operate for extended periods of time (e.g., overnight) without having to transmit the measurements to an external monitor 12. This may provide the patient with greater autonomy and freedom during these periods.

As further shown in FIG. 3, the remote sensor 16 is configured to store a timing marker associated with each average pressure measurement taken (block 68) to permit a time-varying pressure measurement to be later reconstructed and analyzed. For example, for each average pressure measurement computed and stored in memory 44, the remote sensor 16 can be configured to store a timing marker corresponding to the time the actual sensed pressure measurements were taken.

In some embodiments, other information can also be associated with the timing marker to permit other information to be later correlated to the average pressure measurements. In certain embodiments, for example, an activity sensor or accelerometer within the remote sensor 16 can be configured to store patient activity and/or posture measurements within memory 44 along with the average pressure measurements and associated timing markers, allowing the patient's activity and/or posture to be associated with the pressure measurements. Additional information such as heart rate, respiratory rate, barometric pressure, and body temperature could also be stored within memory 44 along with timing markers for later use. For example, a temperature sensor within the remote sensor 16 can be used to sense body temperature at or near the time that the pressure measurements are taken. In some embodiments, the body temperature measurements sensed by the temperature sensor can be associated with the timing markers and stored in memory 44, allowing the pressure measurements to be calibrated, either internally or by another device, based on changes in body temperature.

Once at least one average pressure measurement and associated timing marker is stored in memory 44, the remote sensor 16 may then return to a low-power or sleep state (block 70). Alternatively, and in some embodiments, the remote sensor 16 may further activate the communication circuitry 50 and transmit the average pressure measurements and associated timing markers to a remote device (block 72) for further analysis. In certain embodiments, for example, the remote sensor 16 may activate the communication circuitry 50 and wirelessly transmit the average pressure measurements to the external monitor 12, the pulse generator 14, and/or to another device in wireless communication with the remote sensor 16. Other information such as the patient's body temperature, activity levels, and posture sensed at or near the time of the pressure measurements may also be transmitted along with timing markers associated with these parameters. Once transmitted, the remote sensor 16 may then return to the low-power or sleep state (block 70). The process of waiting in the low-power or sleep state (block 58) until another scheduled time event has lapsed may then be repeated.

The sensing and transmission of pressure measurements can be initiated by an external device in communication with the remote sensor 16. In certain embodiments, for example, a trigger signal sent by the external monitor 12 can be configured to activate the remote sensor 16 and prompt the sensor 16 to transmit the pressure measurements irrespective of whether a scheduled time event has lapsed. In some cases, this may permit the patient or caregiver to receive the pressure measurements on-demand instead of waiting until the next scheduled transmission period programmed within memory 44.

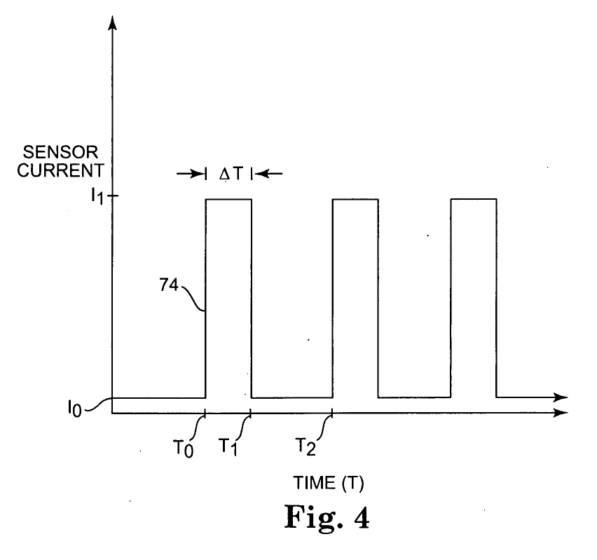

FIG. 4 is a graph showing the operating current 74 of the remote sensor 16 over multiple activation cycles. At time T₀ in FIG. 4, which corresponds to the detection of a scheduled time event programmed in memory 44, the remote sensor 16 is configured to activate the sensing and/or therapy circuitry 46 and begin taking one or more measurements (e.g., arterial blood pressure measurements) within the body. Once activated, the remote sensor 16 takes measurements for a period of time ΔT, which in some embodiments is a parameter pre-programmed within the sensor memory 44. During this time period ΔT, the operating current of the remote sensor 16 increases from an initial magnitude I₀ to a second, higher magnitude I₁ due to the activation of the sensing and/or therapy circuitry 46 and the computation and storage of average pressure measurements within the sensor 16. At time T₁, once the measurements are taken, the remote sensor 16 may then deactivate the sensing and/or therapy circuitry 46 in order to conserve energy within the power supply 52. In certain embodiments, for example, the remote sensor 16 may deactivate the sensing and/or therapy circuitry 46 after a pre-programmed time period (e.g., 5 seconds, 10 seconds, 1 minute, etc.) has elapsed, causing the sensor 16 to revert back to its low-power operating current I₀ in which only the timing circuitry 48 (and in some embodiments other components such as the memory 44) are active.

From time T₁ to T₂, the remote sensor 16 remains in the low-power or sleep state until at such point another scheduled time period programmed within the sensor 16 occurs, causing the sensor 16 to again activate the sensing and/or therapy circuitry 46 and take one or more measurements within the body. As further shown in FIG. 4, the process of activating and deactivating the sensing and/or therapy circuitry 46 in this manner is then repeated for each subsequent interval.

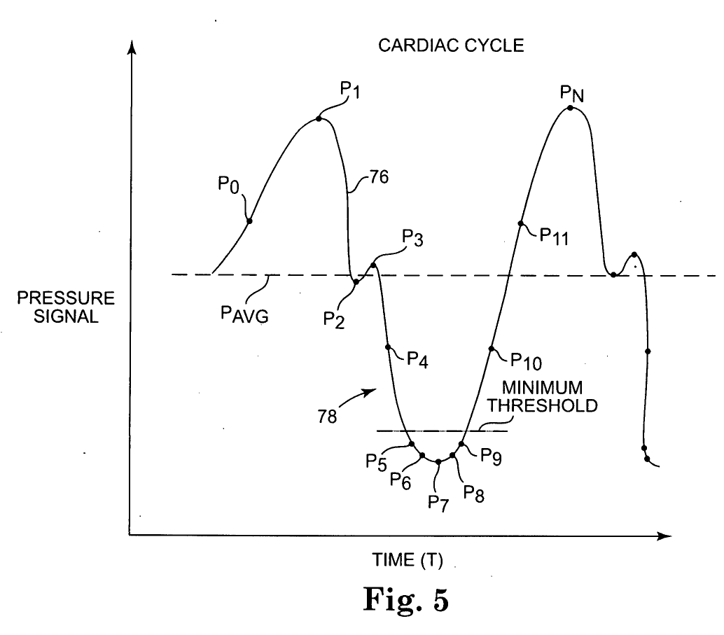

FIG. 5 is a graph showing an illustrative method of taking average pressure measurements from a pressure waveform 74 sensed by the remote sensor 16 over a cardiac cycle. As shown in FIG. 5, the remote sensor 16 can be configured to sample the pressure waveform 74 at discrete time periods P₀, P₁, P₂, . . . , P_{N} over each cardiac cycle. From these sampled pressure measurements P_{N}, the remote sensor 16 may then compute one or more average pressure measurements P_{AVG} representative of the actual pressure during the cycle, during portions of the cycle, or across multiple cycles, similar to that discussed above with respect to block 64 in FIG. 3. In certain embodiments, for example, the remote sensor 16 may sample a number of pressure sensor readings P_{N} and then compute a mean pressure measurement P_{AVG} indicating the average pressure over the cardiac cycle or across multiple cardiac cycles.

The average pressure measurement P_{AVG} can be computed based on various criteria programmed within the remote sensor 16. In some embodiments, for example, the remote sensor 16 may compute an average pressure measurement P_{AVG} based on peak-to-peak measurements sensed during each cardiac cycle. With respect to the illustrative pressure waveform 76 depicted in FIG. 5, for example, the remote sensor 16 can be configured to compute an average pressure measurement P_{AVG} based on the actual peak-to-peak pressures (i.e., P₁,P₇) sensed during each cardiac cycle. In some embodiments, the remote sensor 16 is configured to compute an average pressure measurement based on only a portion 78 (e.g., the diastolic portion) of the pressure waveform 76.

In some embodiments, the remote sensor 16 is adapted to sample only those pressures above or below a threshold value programmed within the sensor 16. For example, if during the cardiac cycle the pressure waveform 76 drops below a minimum threshold value, the remote sensor 16 can be configured to compute an average pressure during this period by sampling only those pressures (i.e., P5, P6, P7, P8, and P9) that fall below this threshold.

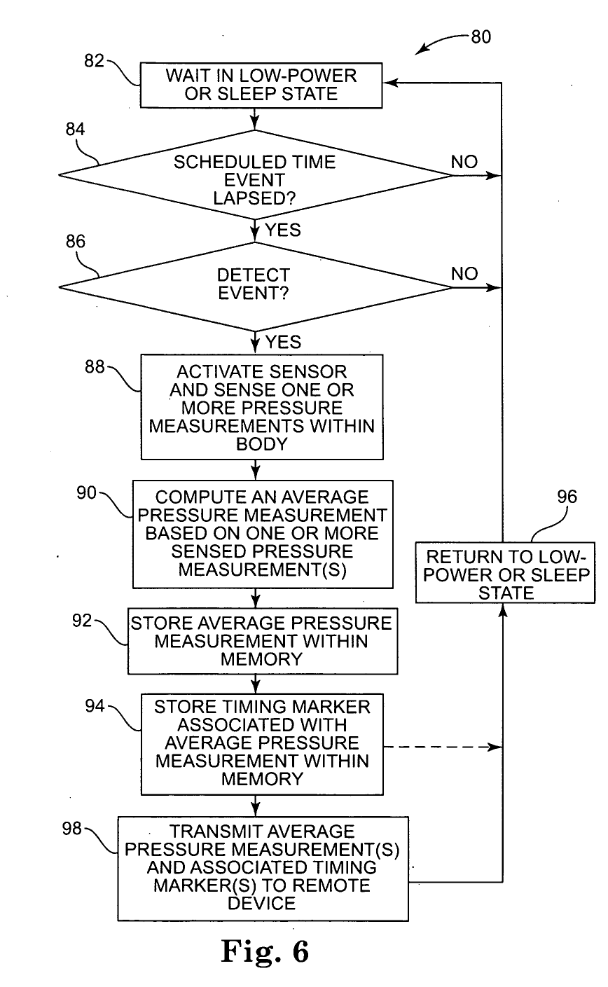

FIG. 6 is a flow chart showing another illustrative method 80 of activating an implantable medical device such as the remote sensor 16 of FIG. 1. The method 80 is similar to the method 56 of FIG. 3, wherein the remote sensor 16 initially waits (block 82) in a low-power or sleep state with one or more components of the sensor 16 either deactivated or placed in a low-power mode. From the low-power or sleep state, the timing circuitry 48 is configured to determine whether a scheduled timing event stored in memory 44 has lapsed (block 84). The determination of whether a scheduled timing event has lapsed can be accomplished, for example, by calling a table of scheduled operating times programmed within memory 44 and comparing the scheduled operating times against the current time and/or date to determine if a scheduled time event has occurred. If the timing circuitry 48 determines that a scheduled time event has not lapsed, the remote sensor 16 continues operation in the low-power or sleep state (block 82).

If the timing circuitry 48 determines that a scheduled time event has occurred, the remote sensor 16 may next determine whether a triggering event has occurred prompting the sensor 16 to activate and take one or more measurements (block 86). In some embodiments, for example, a triggering event such as the detection of the patient's respiratory rate falling below a minimum rate or increasing above a maximum rate may prompt the remote sensor 16 to activate the sensing and/or therapy circuitry 46 and take one or more measurements. The triggering event can be an event sensed by the remote sensor 16 and/or an event sensed by another remote sensor or external device in communication with the sensor 16. Examples of other triggering events could be the detection of pulmonary arterial pressure above or below a certain value, the sensing of temperature or a change in temperature within the body, or the detection of patient activity, posture, or orientation. Other triggering events for activating the remote sensor 16 are also possible.

If a triggering event is not detected at block 86, the remote sensor 16 continues operation in the low-power or sleep state (block 82). Otherwise, if a triggering event has occurred, the remote sensor 16 then activates the sensing and/or therapy circuitry 48, causing the sensor 16 to take one or more pressure measurements within the body (block 88). For example, if the remote sensor 16 receives a signal from a respiratory sensor indicating that the patient's respiratory rate has increased above a certain rate, the sensor 16 may activate the sensing and/or therapy circuitry 46 and begin taking pressure measurements within the body. The respiratory event triggering the activation can then be correlated with the pressure measurements to determine if further treatment is necessary.

Once the sensing and/or therapy circuitry 46 is activated and a number of measurements have been taken, the remote sensor 16 can then compute an average pressure measurement (block 90) based on the one or more sensed measurements, and then store the average pressure measurement within memory (block 92). A timing marker associated with each average pressure measurement may also be stored within memory (block 94) to permit a time-varying pressure measurement to be later reconstructed and analyzed. In some embodiments, other sensed parameters such as temperature, activity, and/or posture may also be stored within memory along with timing markers to correlate the pressure measurements with other parameters. The remote sensor 16 may then either immediately return to the low-power or sleep state (block 96), or alternatively, transmit the average pressure measurement data to a remote device in communication with the sensor 16 (block 98) and then return to the low-power or sleep state (block 96).

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A method carried out by an implantable medical device located within a patient's body, the method comprising:
activating the implantable medical device from a low-power state to an awake state at a scheduled time event programmed within the implantable medical device;
sensing one or more pressure measurements within a pulmonary artery;
computing an average pressure measurement of the pulmonary artery pressure over one or more cardiac cycles based on the one or more sensed pressure measurements;
storing the average pressure measurement within a memory of the implantable medical device;
transmitting the average pressure measurement to a remote device in wireless communication with the implantable medical device; and
returning the implantable medical device to the low-power state.

2. The method of claim 1, wherein transmitting the average pressure measurement occurs at a scheduled time subsequent to taking the pressure measurement.

3. The method of claim 2, wherein transmitting the average pressure measurement occurs in response to a signal from the remote device.

4. A method carried out by an implantable medical device located within a patient's body, the method comprising:
activating the implantable medical device from a low-power state to an awake state at a scheduled time event programmed within the implantable medical device;
sensing one or more pressure measurements within the body;
computing an average pressure measurement based at least in part on the one or more sensed pressure measurements;
storing the average pressure measurement and a timing marker associated with the average pressure measurement within a memory of the implantable medical device; and
returning the implantable medical device to the low-power state.

5. The method of claim 4, further comprising transmitting the average pressure measurement to a remote device in wireless communication with the implantable medical device.

6. The method of claim 4, wherein only timing circuitry within the implantable medical device is activated in the low-power state.

7. The method of claim 4, wherein sensing one or more pressure measurements within the body includes sensing a plurality of pressure measurements over a physiological cycle.

8. The method of claim 7, wherein the physiological cycle is a cardiac cycle, and wherein sensing a plurality of pressure measurements includes sensing a pressure waveform associated with the cardiac cycle.

9. The method of claim 7, wherein computing an average pressure measurement includes computing an average pressure during the physiological cycle.

10. The method of claim 7, wherein computing an average pressure measurement includes computing a minimum or maximum measurement of the pressure during the physiological cycle.

11. The method of claim 7, wherein computing an average pressure measurement includes computing a peak to peak measurement of the pressure during the physiological cycle.

12. The method of claim 7, wherein computing an average pressure measurement includes computing an rms measurement of the pressure during the physiological cycle.

13. The method of claim 7, wherein computing an average pressure measurement includes computing an average pressure measurement across multiple physiological cycles.

14. The method of claim 4, further comprising taking an activity or posture measurement and associating the activity or posture measurement with the timing marker.

15. The method of claim 4, further comprising taking a body temperature measurement and associating the body temperature measurement with the timing marker.

16. The method of claim 4, further comprising activating the implantable medical device from a low-power state to an awake state based on a predetermined event programmed within the implantable medical device.

17. The method of claim 16, wherein the predetermined event is a respiration rate event.

18. The method of claim 16, wherein the predetermined event is a pulmonary artery pressure event.

19. The method of claim 16, wherein the predetermined event is a temperature event.

20. A remote sensor for sensing pressure within a patient's body, comprising:
a power supply adapted to supply power to one or more components of the remote sensor;
a memory unit;
communication circuitry configured to transmit and receive information;
pressure sensing circuitry configured to sense pressure measurements within the body;
timing circuitry configured to activate the sensing circuitry from a low-power state to an active state at a scheduled time period based on one or more timing parameters stored in the memory unit; and
a processor adapted to compute an average pressure measurement based on pressure measurements sensed by the pressure sensing circuitry.
